(19) United States
(12) Patent Application Publication
(10) Pub. No.: US 2019/0328040 A1
(43) Pub. Date: Oct. 31, 2019

(54) Systems and Methods for Buffered Aerosol Drug Delivery

(71) Applicant: Zayd Abdulfuhah Turbi, Irvine, CA (US)

(72) Inventor: Zayd Abdulfuhah Turbi, Irvine, CA (US)

(21) Appl. No.: 16/396,459

(22) Filed: Apr. 26, 2019

Related U.S. Application Data
(60) Provisional application No. 62/664,738, filed on Apr. 30, 2018.

(51) Int. Cl. A24F 47/00; A24B 15/16

(52) U.S. Cl. CPC A24F 47/008; A61K 9/12; A24B 15/167

(57) ABSTRACT

A method for delivering a drug to a user including an electronic cigarette wherein the electronic cigarette itself includes a liquid formulation. The liquid formulation can include at least one drug and at least one biologically acceptable carrier. The at least one drug can be formed with an acid or alcohol and has a first vapor pressure at a first temperature, and is heated by a heating element within the electronic cigarette resulting in the generation of an aerosol suitable for inhaling by the user. Often, the drug can include nicotine, but may also be configured to include cannabinol or can be associated with nicotine replacement therapies, or other medication-assisted therapies. The drugs may also have a salt or co-salt added in order to increase the effectiveness of drug delivery while lowering the need for a higher powered heating element.

### CROSS REFERENCE TO RELATED APPLICATIONS

This application claims the benefit of and priority to U.S. Provisional Application No. 62/664,738, filed Apr. 30, 2018, the entirety of which application is incorporated herein by reference.

### FIELD OF THE INVENTION

Embodiments of the disclosure relate to systems and methods for delivering drugs to a user via a buffered aerosol. More specifically, one embodiment of the disclosure relates to a system, apparatus and method for operating an electronic cigarette to a user comprising a salt or co-salt formulation.

### BACKGROUND

Electronic cigarettes and other vaporizing and vaping devices are an increasingly popular alternative to smoking of traditional combustion cigarettes. Typically, electronic cigarettes convert nicotine-containing liquid into a vapor for inhalation by a user. The electronic cigarette often contains a liquid suitable for aerosolization upon heating, a reservoir to store said liquid, a heating system for heating said liquid, and a power source for providing power to the heating system. The power source is most often battery powered.

An important consideration for electronic cigarettes is obtaining sufficient deep lung delivery of nicotine. Current compositions, devices, and methods may fail to deliver nicotine to the deep lung, and instead primarily deliver nicotine to the oropharynx at the back of the throat or the upper respiratory tract. This may occur for various reasons. For example, nicotine may not be contained in the particle phase of an emitted aerosol, but instead may be a gas that diffuses into the walls of the oropharynx or upper respiratory tract. Alternatively, the nicotine may be in both the particulate and gaseous phases of the aerosol in substantial amounts, but the gaseous fraction may be too high and/or the nicotine may exchange too rapidly between the particulate and gaseous phases, such that it deposits via diffusion of gas into the walls of the oropharynx or upper respiratory tract. In certain instances, this may lead to a “rough” feeling in the walls of the oropharynx of the electronic cigarette user.

Another problem in the field of electronic cigarettes and other vaporizing/vaping devices is obtaining the desired nicotine dose. For example, vaporizing devices may fail to provide consistent dosing from puff to puff. Such as obtaining the same emitted dose of nicotine and the same aerosol particle size from puff to puff. Electronic cigarettes traditionally rely on having an equivalent passage of current through the heating element from puff to puff, at least to the extent the battery technology enables such consistency, and are not equipped to respond to the demands of a particular user. Other common limitations include insufficient aerosol production, slow responsiveness to user demand, risk of overheating, degradation of the substance(s) to be vaporized, inadequate battery power, and/or requirement for frequent recharging of the battery. Collectively, these limitations decrease the effectiveness of these devices. For example, current devices may provide inconsistent heating and/or insufficient aerosol generation, thus failing to simulate the familiar experience of smoking traditional cigarettes or cigars, including the familiar “draw” or ease of vapor production of a combustion cigarette.

It may desirable for users transitioning away from traditional cigarettes to electronic cigarettes is to acquire a high enough level of nicotine in order to be satisfied. However, nicotine delivered via electronic cigarettes is not typically as efficient as traditional cigarettes, leading to lower nicotine intake for users. To compensate, electronic cigarette users may use liquids that contain higher concentrations of nicotine, which can lead to a harsher or rough feeling in the oropharynx (i.e. throat) of the electronic cigarette user.

Past research has been done regarding introducing salt and/or co-salts into nicotine for traditional cigarettes. This research focused on the ability of salts or co-salt formulations to deliver higher nicotine levels with lower amounts of FTC-defined “tar” which typically correlates to the feeling of “harshness” in traditional cigarettes. Thus, there is a need for devices and methods that may provide for a more satisfying experience in the use of vaporizing devices such as electronic cigarettes utilizing salts and/or other formulations.

### SUMMARY OF THE INVENTION

In many embodiments, a method for delivering a drug to a user includes an electronic cigarette wherein the electronic cigarette comprises a liquid formulation including at least one drug, and at least one biologically acceptable carrier, wherein the at least one drug is formed with an acid or alcohol and has a first vapor pressure at a first temperature, and wherein the liquid formulation is heated by a heating element resulting in the generation of an aerosol suitable for inhaling by the user.

A further embodiment includes the at least one drug being nicotine.

An additional embodiment includes the at least one drug being cannabinol.

A still further embodiment includes the at least one drug being associated with a nicotine replacement therapy (“NRT”).

A further embodiment includes the at least one drug being associated with a medication-assisted therapy (“MAT”).

A still additional embodiment includes the at least one drug being salt-based.

A yet further embodiment includes the first vapor pressure is less than 20 millimeters of mercury (mmHg).

A still further embodiment includes the first temperature being approximately 200 degrees Celsius.

In an additional embodiment, the at least first drug also comprises a first melting point and a first boiling point and wherein the difference between the first melting point and the first boiling point is at least 50 degrees Celsius.

In a further embodiment, the heating element operates at a first operating temperature and the first boiling point of the first drug is less than 40 degrees Celsius lower than the operating temperature.

In a variety of embodiments, a liquid storage device including a housing suitable for coupling with an electronic cigarette wherein the coupling provider thermal communication with a heating element within the electronic cigarette, a liquid formulation including at least one drug, and at least one biologically acceptable carrier, wherein the at least one drug is formed with an acid or alcohol and has a first vapor pressure at a first temperature, and wherein the liquid formulation is heated by the heating element resulting in the generation of an aerosol suitable for inhaling by a user.

In a yet further embodiment, the operating temperature is from 150° C. to 250° C.

In an additional embodiment, the operating temperature is from 180° C. to 220° C.

In a still further embodiment, the operating temperature is about 200° C.

In a still further embodiment, the aerosol comprises condensate of the nicotine, cannabinol, NRT, or MAT salt or co-salt.

In an additional embodiment, the aerosol comprises condensate of freebase nicotine.

In a yet further embodiment, the aerosol comprises condensate of the carrier.

In a further embodiment, the aerosol comprises condensate of the acid or alcohol.

In a still additional embodiment, the aerosol comprises condensate in particle sizes from about 0.1 microns to about 5 microns.

In a still further embodiment, the aerosol comprises condensate in particle sizes from about 0.1 microns to about 1 or 2 microns.

In a further embodiment, wherein the aerosol comprises condensate in particle sizes from about 0.1 microns to about 0.7 microns.

In an additional embodiment, the aerosol comprises condensate in particle sizes from about 0.3 microns to about 0.4 microns.

In a yet further embodiment, the acid or alcohol is a carboxylic acid or sugar alcohol.

In a still further embodiment, the acid or alcohol used to form said nicotine, cannabinol, NRT, or MAT salt or co-salt is a sugar alcohol or acid.

In an additional embodiment, the organic acid is monocarboxylic acid, aromatic acid, keto acid, terpene, sugar or a combination thereof.

In a further embodiment, the organic acid comprises terpenoid conjugate, flavor acidulant, or sugar alcohol.

In a further embodiment, the sugar alcohol is one of: is arabinose, arginine, adipic acid, anthranilic acid, ascorbic acid, cannabinol, camphene, cinnamic acid, cinnamyl alcohol, dextrose, deoxyribose, EDTA, erucic acid, ethyl 2-trans-4-cis-decadienoate, eucalyptol, fructose, gallic acid, glutamic acid, glutamine, glycinic acid, indic acid, isoamyl alcohol, oenanthic acid, pelargonic acid, phosphoric acid, phytic acid, proline, lactose, linalool, maltose, mandelic acid, mangoferin, manzanate, menthol, myrcene, nerol, nervonic acid, nicotinic acid, nucleosidic acid, mannitol, pectin, limonene, pinene, punicic acid, ribose, sabinene, sativic acid, sebacic acid, sorbitol, sucralose, tocopherols, thiamine, thymol, vanillin, xylose, xylitol and ethyl maltol.

In a still additional embodiment, the acid or alcohol used to form the nicotine, cannabinol, NRT, or MAT salt or co-salt is anthranilic acid.

In a still further embodiment, the acid or alcohol used to form the nicotine, cannabinol, NRT, or MAT salt or co-salt is oenanthic acid.

In a yet further embodiment, the acid or alcohol used to form the nicotine, cannabinol, NRT, or MAT salt or co-salt is nicotinic acid.

In an additional embodiment, the acid or alcohol used to form the nicotine, cannabinol, NRT, or MAT salt or co-salt is ascorbic acid.

In a further embodiment, the acid or alcohol used to foam the nicotine, cannabinol, NRT, or MAT salt or co-salt is cinnamic acid.

In a still further embodiment, the acid or alcohol used to form the nicotine, cannabinol, NRT, or MAT salt or co-salt is glutamic acid.

In an additional embodiment, the nicotine, cannabinol, NRT, or MAT salt or co-salt comprises nicotine, cannabinol, NRT, or MAT nicotinate.

In a further embodiment, the nicotine, cannabinol, NRT, or MAT salt or co-salt comprises nicotine, cannabinol, NRT, or MAT anthranilate.

In a yet further embodiment, the nicotine, cannabinol, NRT, or MAT salt or co-salt comprises nicotine, cannabinol, NRT, or MAT ascorbate.

In a still additional embodiment, the nicotine, cannabinol, NRT, or MAT salt or co-salt comprises nicotine, cannabinol, NRT, or MAT oenanthate.

In a still further embodiment, the nicotine, cannabinol, NRT, or MAT salt or co-salt comprises nicotine, cannabinol, NRT, or MAT glutamate.

In a yet further embodiment, the nicotine, cannabinol, NRT, or MAT salt or co-salt comprises nicotine, cannabinol, NRT, or MAT cinnamate.

In an additional embodiment, the liquid carrier comprises glycerol, propylene glycol, trimethylene glycol, water, ethanol or combinations thereof.

In a further embodiment, the liquid carrier comprises propylene glycol and vegetable glycerin.

In a still further embodiment, the liquid carrier comprises 20% to 50% of propylene glycol and 50% to 80% of vegetable glycerin.

In an additional embodiment, the liquid carrier comprises 30% propylene glycol and 70% vegetable glycerin.

In a further embodiment, the nicotine, cannabinol, NRT, or MAT salt or co-salt is in an amount that forms about 0.5% to about 20% nicotine, cannabinol, NRT, or MAT in the inhalable aerosol.

In a yet further embodiment, the nicotine, cannabinol, NRT, or MAT salt or co-salt is in an amount that forms about 1% to about 20% nicotine, cannabinol, NRT, or MAT in the inhalable aerosol.

In a still additional embodiment, the liquid formulation has a nicotine, cannabinol, NRT, or MAT concentration of about 1% (w/w) to about 25% (w/w).

In a still further embodiment, the liquid formulation has a nicotine, cannabinol, NRT, or MAT concentration of about 1% (w/w) to about 20% (w/w).

In a further embodiment, the liquid formulation has a nicotine, cannabinol, NRT, or MAT concentration of about 1% (w/w) to about 18% (w/w).

In an additional embodiment, the liquid formulation has a nicotine, cannabinol, NRT, or MAT concentration of about 1% (w/w) to about 15% (w/w).

In a yet further embodiment, the liquid formulation has a nicotine, cannabinol, NRT, or MAT concentration of about 4% (w/w) to about 12% (w/w).

In a still further embodiment, the liquid formulation has a nicotine, cannabinol, NRT, or MAT concentration of about 4% (w/w).

In an additional embodiment, the liquid formulation has a nicotine, cannabinol, NRT, or MAT concentration of about 2% (w/w).

In a further embodiment, the formulation further comprises a flavorant.

In a still additional embodiment, the formulation is non-corrosive to an electronic cigarette.

In a further embodiment, the acid or alcohol is stable at and below operating temperature or about 200° C.

In a yet further embodiment, the acid or alcohol does not decompose at and below operating temperature or about 200° C.

In an additional embodiment, the acid or alcohol does not oxidize at and below operating temperature or about 200° C.

In a still further embodiment, the formulation is non-corrosive to the electronic cigarette.

In a further embodiment, the formulation is non-toxic to a user of the electronic cigarette.

In a still additional embodiment, the formulation further comprises one or more additional nicotine, cannabinol, NRT, or MAT salts or co-salts in a biologically acceptable liquid carrier suitable for generating the inhalable aerosol upon heating.

In a further embodiment, a second acid used to form the additional nicotine, cannabinol, NRT, or MAT salt or co-salt is selected from the group consisting of anthranilic acid, ascorbic acid, sativic acid, indic acid, oenanthic acid, nicotinic acid, cinnamic acid, and glutamic acid.

In an additional embodiment, a nicotine, cannabinol, NRT, or MAT salt or co-salt liquid formulation in an electronic cigarette for generating an inhalable aerosol upon heating in the electronic cigarette, the formulation in the cigarette comprising a nicotine, cannabinol, NRT, or MAT salt or co-salt in a biologically acceptable liquid carrier wherein an acid or alcohol used to form said nicotine, cannabinol, NRT, or MAT salt or co-salt is characterized by vapor pressure >20 mmHg at 200° C.

In a still further embodiment, the formulation comprises a nicotine, cannabinol, NRT, or MAT salt or co-salt liquid formulation in an electronic cigarette for generating an inhalable aerosol upon heating in the electronic cigarette, the formulation in the cigarette comprising a nicotine, cannabinol, NRT, or MAT salt or co-salt in a biologically acceptable liquid carrier wherein an acid or alcohol used to form said nicotine, cannabinol, NRT, or MAT salt or co-salt is characterized by vapor pressure of about 20 to 200 mmHg at 200° C.

In a yet further embodiment, the formulation comprises a nicotine, cannabinol, NRT, or MAT salt or co-salt liquid formulation in an electronic cigarette for generating an inhalable aerosol upon heating in the electronic cigarette, the formulation in the cigarette comprising a nicotine, cannabinol, NRT, or MAT salt or co-salt in a biologically acceptable liquid carrier wherein an acid or alcohol used to form said nicotine, cannabinol, NRT, or MAT salt or co-salt is further characterized by a melting point <160° C., a boiling point >160° C., and at least a 50-degree difference between the melting point and the boiling point.

In a still additional embodiment, the formulation comprises a nicotine, cannabinol, NRT, or MAT salt or co-salt liquid formulation in an electronic cigarette for generating an inhalable aerosol upon heating in the electronic cigarette, the formulation in the cigarette comprising a nicotine, cannabinol, NRT, or MAT salt or co-salt in a biologically acceptable liquid carrier wherein an acid or alcohol used to form said nicotine, cannabinol, NRT, or MAT salt or co-salt is further characterized by a melting point at least 40 degrees lower than an operating temperature of the electronic cigarette, a boiling point no more than 40 degrees lower than the operating temperature of the electronic cigarette, and at least a 50-degree difference between the melting point and the boiling point.

In a still further embodiment, the electronic cigarette heats the nicotine salt or co-salt formulation to an operating temperature from 150° C. to 250° C.

In an additional embodiment, the electronic cigarette heats the nicotine salt formulation to an operating temperature from 180° C. to 220° C.

In a yet further embodiment, the electronic cigarette heats the nicotine salt or co-salt formulation to an operating temperature of about 200° C.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1A shows an exploded, partial cross-section view of an exemplary electronic cigarette suitable for use with one or more embodiments of the invention.

FIG. 1B shows the electronic cigarette of FIG. 1A assembled, in accordance with one or more embodiments of the invention.

FIG. 2 shows an exemplary electronic cigarette, in accordance with one or more embodiments of the invention.

FIG. 3 shows an exemplary vaporizing device, in accordance with one or more embodiments of the invention.

FIG. 4 shows a portion of an exemplary vaporizing device, in accordance with one or more embodiments of the invention.

FIG. 5A shows an exemplary quinic compound for use in a formulation in accordance to one or more embodiments of the invention.

FIG. 5B shows an exemplary nicotinic compound for use in a formulation in accordance to one or more embodiments of the invention.

FIG. 5C shows an exemplary sorbic compound for use in a formulation in accordance to one or more embodiments of the invention.

FIG. 5D shows an exemplary cinnamic compound for use in a formulation in accordance to one or more embodiments of the invention.

FIG. 5E shows an exemplary oxybenzoic compound for use in a formulation in accordance to one or more embodiments of the invention.

FIG. 5F shows an exemplary acetylic and acetylcysteine compounds for use in a formulation in accordance to one or more embodiments of the invention.

### DETAILED DESCRIPTION

Various embodiments of the disclosure relate to a method and/or device configured to deliver at least one drug to a user through the inhalation of a vaporized liquid. In general, a formulation containing various salt and/or co-salt compounds may be utilized by an electronic cigarette to vaporize the liquid through heating to generate a vapor that may be inhaled by the user to deliver at least one drug contained within the liquid formulation. The use of various salts, co-salts, and/or alternative chemicals may provide for a more robust method of delivering higher concentrations of drugs to a user without the need for a higher-wattage heater, yielding practical benefits including, but not limited to, allowing for the use of a reduced-size electronic cigarette, and/or to increase the chances of success for users going through nicotine replacement therapies.

Many popular tobacco products utilize nicotine salts in tandem with natural free base nicotine to increase its safe digestibility. The salts can act to mitigate the off-target effects of nicotine while still delivering the euphoric and stimulant effects of the alkaloid. This principle can be applied to nicotine replacement therapies as well as to medication-assisted therapies including, but not limited to, cannabinoids, opioids, and other inhalable medications.

The production and use of salts in traditional cigarettes has been disclosed previously as taught in U.S. Pat. No 4,830,028 filed Feb. 10, 1987 entitled “Salts Provided from Nicotine and Organic Acid as Cigarette Additives” which is hereby incorporated in its entirety, with emphasis on the production and ratios of organic acids and formed salts. Traditionally, salts have been produced through the combination of at least one ingredient and an at least one organic acid. For traditional cigarettes, nicotine salts were provided from nicotine and organic acids, which were then incorporated into cigarettes as an additive. In these embodiments, the molar ratio of nicotine to organic acid is typically 1:3, however other ratios (such as 1:1 and 1:3) could also be utilized.

Organic acids that may be utilized to generate salts for use in electronic cigarettes may be comprised of any organic acids or organic compounds that behave as Lewis acids when contacted with nicotine or other compounds. Preferred organic acids are those acids which form salts with the selected compound in a 1:1, 2:1 or 3:1 molar ratio (organic acid:selected compound). Examples of organic acids that may be useful in practicing the present invention are aromatic acids including benzoic-type acids and substituted benzoic-type acids, hydroxyacids, heterocyclic acids, terpenoid acids, sugar acids such as the pecticacids, amino acids, cycloaliphatic acids, dicarboxylicacids, aliphatic acids, keto acids, and the like. Other organic acids that may be utilized are the straight-chain and branched-chainaliphatic acids, more preferably the aliphatic monocarboxylic acids, and especially the keto aliphatic monocarboxylic acids. Examples of suitable organic acids include formic, acetic, propionic, isobutyric, butyric, alpha-methylbutyric, isovaleric, beta-methylvaleric, caproic, 2-furoic, phenylacetic, heptanoic, octanoic, nonanoic, malic, citric, oxalic, malonic, glycolic, succinic, ascorbic, tartaric, fumaric and pyruvic acids, as well as the lower fatty acids (i.e., having carbon chains less than C12) and the higher fatty acids (i.e., having carbon chains of C12 to C20), and other such acids. In certain embodiments, a preferred organic acid may be a gamma keto aliphatic acid such as levulinic acid (CH₃COCH₂CH₂COOH). Useful organic compounds which exhibit an acid character and which form salts with selected compounds can include the phenolics such as guaiacol, vanillin, protocatechualdehyde, and the like.

The nicotine/organic acid salts are prepared using techniques generally known to those skilled in the art. Preferably, nicotine/organic acid salts are provided by contacting the selected compound with at least the stoichiometric amount of the organic acid necessary to form the particular salt under conditions sufficient to form the salt. In many embodiments, salts provided from selected compounds and organic acids are essentially non-volatile at conditions under which the articles are manufactured and stored, however, specific properties of the various salts can vary. For example, nicotine/organic acid salts having straight-chain or branched-chain aliphatic acids having less than 20 carbon atoms generally have a molar ratio of 1:3 (nicotine:acid), and generally are liquid in form at ambient conditions. In fact, many salts produced in this manner form a liquid at ambient temperatures. Such liquids may be utilized as formulations within an electronic cigarette or other such vaporizing systems.

### Electronic Cigarette Devices

The present disclosure is not limited to any particular vaporization/vaping device or vaporization method. The compositions described herein generally may be used, for example, in any electronic cigarette, cigar, vaping device, or other vaporization device, including disposable and/or rechargeable devices, and commercially-available devices, as well as any suitable containers for compositions used for aerosol generation.

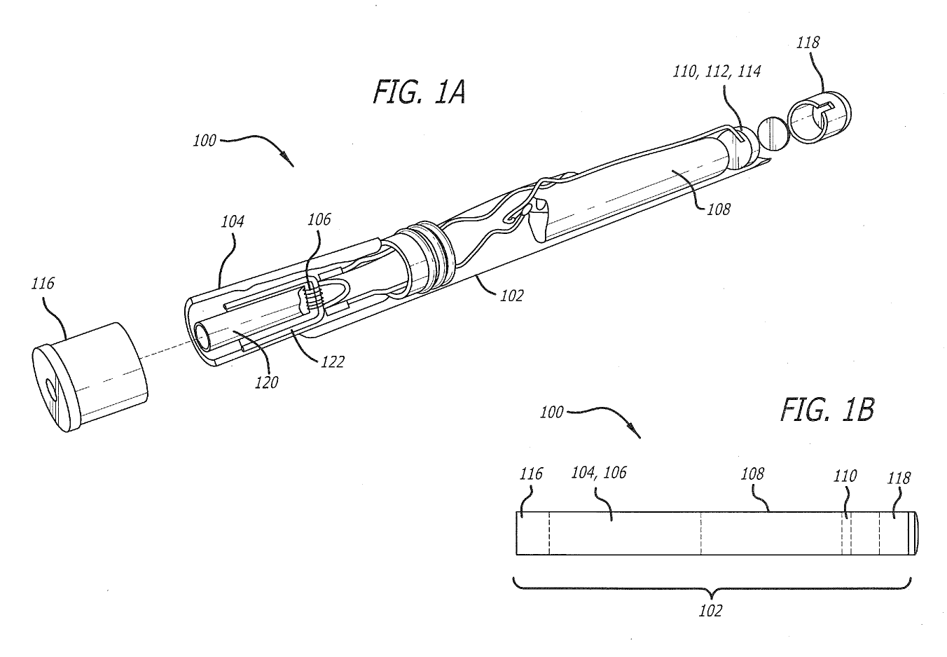

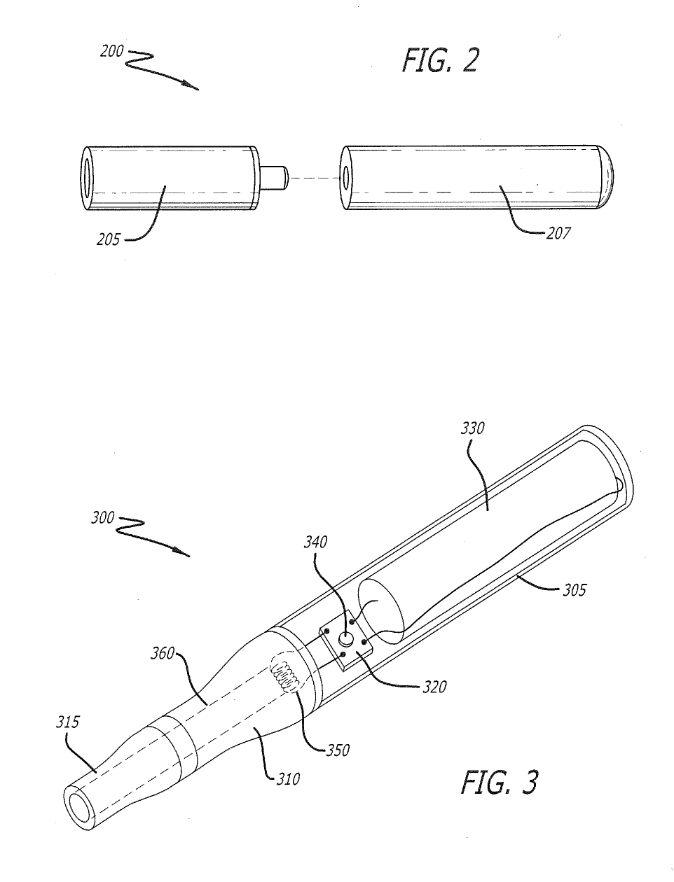

Referring to FIGS. 1A-1B, an exploded, partial cross-section view of an exemplary electronic cigarette 100 suitable for use with one or more embodiments of the invention is shown. The electronic cigarette 100 may comprise a housing 102 that completely covers all internal components of the electronic cigarette 100, as shown in FIG. 1B. While FIGS. 1A and 1B illustrate an exemplary combination of internal components, vaporizing devices according to the present disclosure need not include each and every component shown. The housing 102 may be flexible and/or resilient along at least a portion of the housing 102, e.g., the entire length of the housing 102. The housing 102 may be covered by a paper label, e.g., to simulate the appearance and/or feel of a traditional cigarette. In some embodiments, the housing 102 may comprise a two (or more) piece assembly. For example, the housing 102 may comprise two or more components configured to be disassembled for purposes of charging or replacing a battery and/or replacing a liquid containing cartridge (see, e.g., FIG. 2, discussed below).

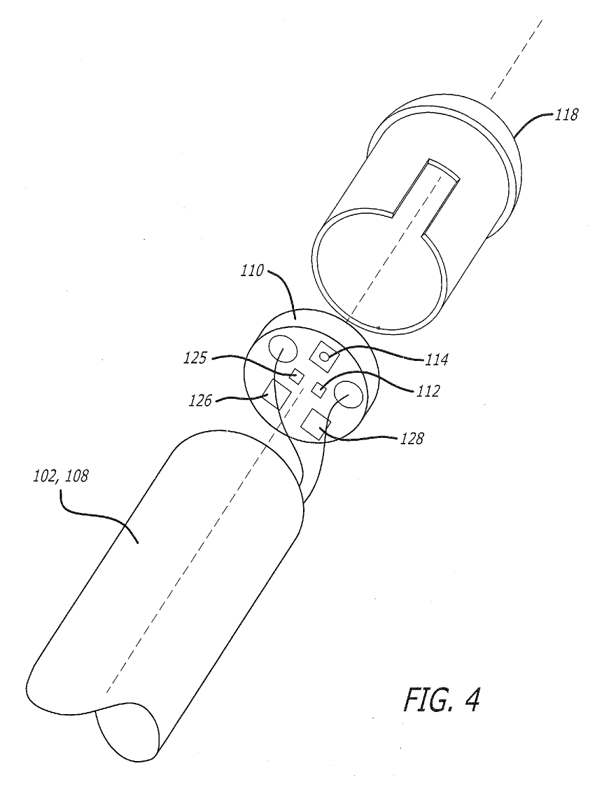

Referring to FIGS. 1 and 4, the internal components of the electronic cigarette 100 may include one or more of a reservoir 104, a heating element 106, a battery 108, an integrated circuit 110, a processor or microprocessor 125, memory 126, a transmitter 128, at least one sensor 112, and/or at least one light source 114, e.g., a light-emitting diode (LED). It is contemplated that features with respect to a battery, operation of a battery, a microprocessor, and/or transmitting or recording information regarding power characteristics or inhalation characteristics may be utilized in order to increase efficiency, lifespan, and/or to store customer preferences.

The electronic cigarette 100 may include a mouthpiece 116 which may be inserted in a first end of the housing 102 and a tip portion 118 which can be inserted in a second end of the housing 102. The outermost surface of the first end of the housing 102 (e.g., outside of the label) may include a coating to protect against moisture from the user's mouth. The tip portion may include at least one air inlet, e.g., a notch in the tip portion 118, and may be at least partially transparent to allow light to pass through to simulate the natural burn of a traditional cigarette. The mouthpiece 116 may include an outlet in communication with a conduit 120 through the reservoir 104, e.g., for inhaling a vaporized composition. It is contemplated that the LED color and placement may be altered as a design choice based on user preference.

The reservoir 104 may comprise an absorbable material, e.g., cotton fiber or other fibrous matrix, that includes a liquid composition absorbed therein as described above. For example, the fiber may be saturated with a liquid comprising nicotine or other salt and/or co-salt containing compound according to the present disclosure. Alternatively, the reservoir 104 may be comprised of a liquid composition that may be in thermal communication with the heating element 106. In further embodiments, the reservoir 104 may comprise part of an aerosol assembly that includes the heating element 106 coupled to a wick 122, for example, wherein the wick 122 may absorb or adsorb liquid from the fiber. Inhalation by a user at the outlet of the mouthpiece 116 may lower the pressure in the housing 102, where in the negative pressure may be detected by the sensor 112. The sensor 112 may cause the heating element 106 to turn on, thus generating heat, and causing the liquid absorbed by the wick 122 to vaporize. The vaporized composition may be drawn through the conduit and condense into an aerosol, e.g., via spontaneous condensation, which exits the electronic cigarette 100 via the outlet in the mouthpiece 116 via the conduit 120, e.g., into the user's lungs. Any features with respect to aspects or components of a vaporizing unit. e.g., a reservoir, a wick, a heating element, and/or other components used for vaporization, may be used according to the present disclosure.

In some embodiments, for example, the electronic cigarette may include a filter section in addition to, or as an alternative to, the mouthpiece 116. The filter section may include a porous material Such as a membrane, a fibrous matrix, or disc that allows vapor to pass there through to simulate the experience of inhaling through a traditional cigarette filter. Any of the features of a filter as taught in the art may be used according to the present disclosure. For example, the filter section may include an acidic fiber. In some embodiments, the filter section may include one or more openings for passage of vapor in combination with, or as an alternative to, the porous material. It is contemplated that many embodiments may utilize filters or other similar features that better recreate the sensation of traditional cigarettes when used with nicotine replacement therapies.

Other exemplary vaporizing devices that may use compositions as described herein for vapor and aerosol generation are shown in FIGS. 2 and 3, each of which may include any combination of the internal components of the electronic cigarette 100 discussed above. FIG. 2 shows an exemplary rechargeable electronic cigarette 200 comprising a cartridge unit 205 and a battery unit 207 that may be connected for use, e.g., via complementary threaded portions or other mating elements, and disconnected for replacement, recharging, or repair as needed. For example, the cartridge unit 205 may include a vaporization unit comprising one or more of a reservoir 104, a heating element 106, a wick 122, and/or a conduit 120; and the battery unit 207 may include one or more of a battery 108, an integrated circuit 110, sensor(s) 112, and/or light source(s) 114. In sonic embodiments, the battery unit 207 may include a rechargeable battery, and the cartridge unit 205 may include a refill valve or tank for receiving a liquid composition as described above. In some embodiments, the cartridge unit 205 may be configured for one-time use, such that once the liquid composition in a first cartridge unit has been depleted, a second, replacement cartridge unit may be connected to the battery unit 207 for use.

FIG. 3 shows an exemplary vaping device 300 comprising a base 305, a liquid tank 310, and a mouthpiece 315. The base 305 may house a battery 330, e.g., a rechargeable battery, operably coupled to a printed circuit board (PCB) assembly 320 and a heating element, e.g., heating wire 350. The tank 310 may include a composition as described above, e.g., to generate aerosols upon application of heat to the composition from the heating wire 350. In some embodiments, the vaping device 300 may include an actuator such as a power button 340 to initiate, control, and/or terminate heat supplied to the heating wire 350. Additionally or alternatively, the vaping device may include a sensor, such as, but not limited to, the sensor 112 of electronic cigarette 100, for controlling the supply of heat upon detecting certain conditions or thresholds. An inner portion of the tank 310 may define an airway 360 extending through the mouthpiece 315 for generation of condensation aerosol, and passage of the aerosols to a user for inhalation. The tank 310 may be refillable, e.g., via a suitable refill valve or inlet, or replaceable to replenish the vaporing device 300 with the composition (or another composition having, e.g., different flavors and/or concentrations of nicotine), as needed.

The integrated circuit(s) 110 may be configured to control and/or receive information from one or more electronic components of the vaporizing device, such as, e.g., the sensor(s) 112, the light source(s) 114, the memory 126, and/or the transmitter(s) 128. Suitable types of integrated circuits 110 according to the present disclosure may include, but are not limited to, analog, digital, and mixed signal integrated circuits, application-specific integrated circuits (ASICs), and microprocessors. In some embodiments, one or more sensor(s) 112 and/or one or more light source(s) 114 may be directly coupled to the integrated circuit 110, as shown in FIG. 4, or may otherwise be operably coupled to the integrated circuit 110 to transmit and receive information. Examples of sensors 112 suitable for the present disclosure include pressure sensors, accelerometers or other motion sensors, flow rate sensors, heat sensors, moisture sensors, temperature sensors, electrical current and/or resistance sensors, and other devices and components for detecting various environmental, chemical, or biological conditions or thresholds. In addition or alternatively, the integrated circuit 110 may include the microprocessor 125, the memory 126, and/or one or more transmitters 128, e.g., directly coupled to the integrated circuit 110, as shown in FIG. 4, or otherwise operably coupled to the integrated circuit 110. The integrated circuit 110, the sensor(s) 112, the light source(s) 114, the microprocessor 125, the memory 126, and/or the transmitter(s) 128 may be coupled via a printed circuit board. The shaft of the tip portion 118 may have an inside diameter larger than the outside diameter of the integrated circuit 110 so that the integrated circuit 110 may be held securely within the shaft.

Upon inhalation of the vaporizing device, for example, a pressure sensor 112 may detect a pressure level and/or change in pressure within the vaporizing device (e.g., electronic cigarette 100 or 200, or vaping device 300), which may in turn control one or more other components of the vaporizing device. For example, information from the pressure sensor 112 may trigger control of the battery 108 and/or light source(s) 114 through the integrated circuit 110. A change in pressure detected within the vaporizing device may prompt the battery 108 to supply power to the heating element, thus heating a liquid composition within the vaporizing device to produce a vapor. In some embodiments, the vaporizing device may include more than one pressure sensor 112, or a combination of different sensors, e.g., including a pressure sensor 112 and one or more other sensors.

The vaporizing device may be configured to vaporize effectively a vaporization Substance (e.g., a liquid and/or solid composition to be vaporized, such as the compositions described herein) without excessive thermal decomposition of the substance. To this end, the effective voltage and resistance of the vaporizing device may be chosen so as to generate a desired quantity of vaporization, e.g., a desired amount of the Substance in aerosol form (e.g., 0.25 mg. 0.5 mg, 0.75 mg. 1 mg, 2 mg, 3 mg. 4.5 mg, 7 mg, 10 mg, 15 mg, 20 mg. 30 mg. or 50 mg) per unit time (e.g., 0.25 seconds, 0.5 seconds, 0.7 seconds, 1 second, 2 seconds, 3 seconds, or 4 seconds). The effective voltage and resistance of the vaporizing device may further be chosen so as to generate a desired quantity of vaporization without excessive thermal degradation.

This may be achieved, for example, by having the integrated circuit 110 direct the battery 108 to pass sufficient current through the heating element 106 for an initial amount of time to effectively initiate rapid vaporization, and thereafter direct the battery 108 to pass a lesser current through the heating element 106 so as to avoid overheating of the heating element 106 or vaporization substance and associated thermal degradation. Thermal degradation may be of particular concern in electronic cigarettes or vaping devices when vaporizing thermally-labile flavoring agents, salts, co-salts, and/or other substances which may act to decrease the vapor pressure.

Certain materials may affect the performance and/or stability of compositions used to generate aerosols. In addition to the various components of a vaporizing device or containers for housing device components of a vaporizing device, the materials used in manufacturing the device, materials in the device itself, and/or materials used in containers for housing or storing the composition used to generate the aerosol may impair the performance and/or stability of the composition. Certain metals or metal alloys, for example, may catalyze, accelerate, or otherwise promote degradation of various chemical compounds. Thus, devices, device components, and containers suitable for the present disclosure may include materials that do not catalyze the degradation of one or more components of the composition Such as nicotine, ion pairing agent(s), carrier solvent(s), and/or other components.

For example, embodiments of the present disclosure include disposable and refillable devices such as liquid loaded devices, cartomizers (e.g., for housing a liquid and configured to mate with, or otherwise compatible with, a power source such as a battery or battery unit for vaporizing the liquid), and bottles and other containers used for storage of a liquid (e.g., used to fill a separate vaporizing/vaping device). Those devices, bottles, and containers may comprise materials that do not promote degradation of the composition, and may not comprise materials that are detrimental to the performance and/or stability of the composition. By way of example and not limitation, the present disclosure includes vaporizing devices, cartomizers, and containers that do not comprise quantities of metals sufficient to catalyze the degradation of nicotine and/or other components of the composition. Exemplary metals that are not in contact with the composition may include, but are not limited to, brass and copper. In some embodiments, the device(s) or various components of the device(s) may lack materials that act as catalysts to degrade salts, co-salts and/or other components of the composition. In some embodiments, the device(s) or various components of the device(s) may be configured to prevent contact between the composition and any materials that may act as catalysts to degrade the composition.

### Formulations

Many different formulations are disclosed herein that may be used in conjunction with various embodiments of electronic cigarettes and/or other vaporizing devices including, but not limited to, those discussed in FIGS. 1A-4. In many embodiments, the formulations will be provided in a liquid form that can be vaporized when exposed to a first temperature to generate an aerosol for inhalation by a user. In additional embodiments, the formulation can be an aerosol buffer formulation of natural triglyceride, flavor acidulant, and sugar alcohol salts such as (but not limited to) arabinose, arginine, adipic acid, anthranilic acid, ascorbic acid, cannabinol, camphene, cinnamic acid, cinnamyl alcohol, dextrose, deoxyribose, EDTA, erucic acid, ethyl 2-trans-4-cis-decadienoate, eucalyptol, fructose, gallic acid, glutamic acid, glutamine, glycinic acid, indic acid, isoamyl alcohol, oenanthic acid, pelargonic acid, phosphoric acid, phytic acid, proline, lactose, linalool, maltose, mandelic acid, mangoferin, manzanate, menthol, myrcene, nerol, nervonic acid, nicotinic acid, nucleosidic acid, mannitol, pectin, limonene, pinene, punicic acid, ribose, sabinene, sativic acid, sebacic acid, sorbitol, sucralose, tocopherols, thiamine, thymol, vanillin, xylose, xylitol and/or ethyl maltol. The ratios can often range from 1:1 up to 1:3 with combinations of the various corresponding acid or alcohol conjugates.

As discussed above, the heating element of an electronic cigarette or other vaporizer may operate at a regular operating temperature. In many embodiments, the operating temperature of the heating element can be a range of temperatures that can vary based on either available power levels and/or input given from the microprocessor (derived from user input such as a button push). In a variety of embodiments the formulation comprises a liquid which has a boiling point below the operating temperature of the heating element. In further embodiments, the formulation may be a solvent having a melting point which is at a lower temperature than the boiling point of the formulation.

In a variety of embodiments, the operating temperature of the heating element may be within a range of 150° C. to 250° C. In further embodiments, the operating temperature range may only be from 180° C. to 220° C. In certain additional embodiments, the operating temperature can be approximately 200° C.

Certain embodiments of the disclosure also comprise a cartomizer for an electronic cigarette comprising a liquid formulation comprising a salt or co-salt compound and a biologically acceptable liquid carrier, a fluid storage compartment, and an atomizer comprising a heating element in fluid communication with the cannabinoid liquid formulation. Certain additional embodiments of this disclosure also provide for a cartridge for use in an electronic cigarette comprising a liquid formulation comprising a salt and/or co-salt compound and a biologically acceptable liquid carrier.

The formulations described herein vary in concentration. In some formulations, a dilute concentration of nicotine, cannabinol, salt, co-salt, or other therapeutic compounds in the carrier can be utilized. In some formulations, a less dilute concentration in the carriers may be utilized. In some formulations the concentration of nicotine, cannabinol, salt, co-salt, or other therapeutic compounds formulation is about 1% weight per weight (w/w) to about 25% (w/w). In some formulations the concentration of nicotine, cannabinol, salt, co-salt, or other therapeutic compounds in the formulation may be about 1% (w/w) to about 20% (w/w). In some other formulation embodiments the concentration of nicotine, cannabinol, salt, co-salt, or other therapeutic compounds in the formulation can be about 1% (w/w) to about 18% (w/w). In some further formulations the concentration of nicotine, cannabinol, salt, co-salt, or other therapeutic compounds in the formulation can be about 1% (w/w) to about 15% (w/w). In additional embodiments the concentration of nicotine, cannabinol, salt, co-salt, or other therapeutic compounds in the formulation is about 4% (w/w) to about 12% (w/w). In yet more formulations the concentration of nicotine, cannabinol, salt, co-salt, or other therapeutic compounds in the formulation can be about 4% (w/w). In some embodiments of the formulations the concentration of nicotine, cannabinol, salt, co-salt, or other therapeutic compounds in the formulation is about 2% (w/w). In some formulations, a less dilute concentration of one nicotine, cannabinol, salt, co-salt, or other therapeutic compounds may be used in conjunction with a more dilute concentration of a second formulation comprising one of nicotine, cannabinol, salt, co-salt, or other therapeutic compounds. In some formulations, the concentration of nicotine, cannabinol, salt, co-salt, or other therapeutic compounds in the first cannabinoid formulation is about 1% to about 20% in the inhalable aerosol.

It is contemplated that many of the formulation embodiments can contain a flavorant. It is well known in the art that the addition of flavorants can provide multiple options for customers of electronic cigarettes. Flavorants may be added as desired by either the manufacturer of the formulations or by a user.

In a number of embodiments, the acid or alcohol of the formulation is compositionally stable and does not decompose or oxidize at various temperatures including at or below 200° C. It would also be obvious to those skilled in the art that the formulations are composed such that they are non-toxic to users of electronic cigarettes or other vaporizers.

It is also understood that the various formulations may be utilized in an electronic cigarette to generate an inhalable aerosol upon heating under various vapor pressures. In certain formulations, the resulting aerosol may be characterized by vapor pressures greater than 20 millimeters of mercury (mmHg). In additional embodiments, the formulation may be characterized by vapor pressure between 20 to 200 mmHg. Such vapor pressures can be obtained at operating temperatures of about 200° C.

Aerosol generated from the formulations can yield particles of various sizes. Those skilled in the art will understand that particle size can be related to absorption of the aerosol within the bloodstream of the user. In many embodiments, the aerosol may comprise condensate in particle sizes from about 0.1 to 5 microns in diameter. In further embodiments, the particle sizes may vary from between 0.1 to 1 or 2 microns. In yet additional embodiments, the particles created may be smaller in scale comprising diameters of 0.1 to 0.7 microns. In still more embodiments, the particle sizes may be more uniform with diameters of 0.3 to 0.4 microns.

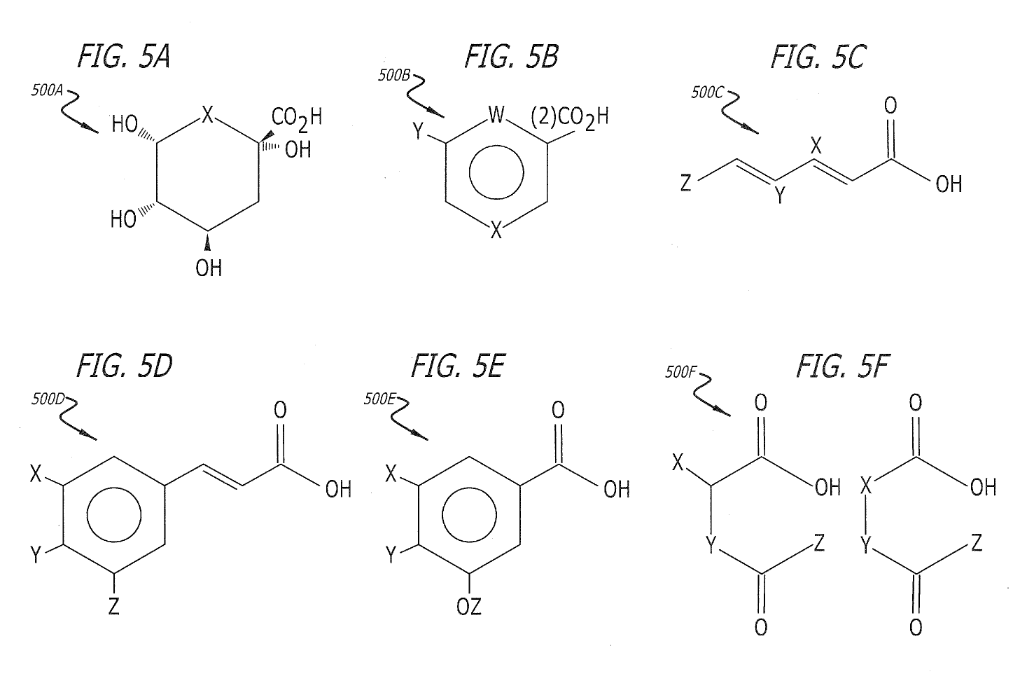

FIGS. 5A-5F shows exemplary compounds for use in a formulation in accordance to one or more embodiments of the invention. It is understood that formulations for use in electronic cigarettes or other vaporizers may contain any of a variety of compounds. By way of example and not limitation, the typical anthranillic and/or oenanthic acids can be replaced with vanillic and/or quinic acids. Additionally, salts and/or co-salts can be produced by exposing a base compound to an organic acid and/or alcohol. In certain embodiments, such acids and/or alcohols may be carboxylic acid or sugar alcohol. In further embodiments, the organic acid may be a monocarboxylic acid, aromatic acid, keto acid, terpene, sugar or a combination thereof. In still yet further embodiments the organic acid may comprise a terpenoid conjugate or flavor acidulant.

FIG. 5A shows a quinic compound 500A in accordance with an embodiment of a formulation of the invention. It should be understood by those skilled in the art that molecules and/or compounds labelled with a W, X, Y or Z are considered to be variable moieties and can be adjusted as needed. Similarly, FIG. 5B shows a nicotinic compound 500B that can be utilized in various formulations according to certain embodiments of the invention. Likewise, FIG. 5C is an exemplary sorbic compound 500C that may be used in accordance with embodiments of the invention. FIG. 5D comprises a cinnamic (or caffeic) compound 500D that can be used in accordance with embodiments of the invention. FIG. 5E shows an oxybenzoic compound 500E suitable for use in generating formulations in accordance with embodiments of the invention. Finally, FIG. 5F shows acetylic and acetylcysteine compounds 500F suitable for use in generating formulations in accordance with embodiments of the invention.

In a further embodiments, the sugar alcohol utilized in developing formulations may be one of arabinose, arginine, adipic acid, anthranilic acid, ascorbic acid, cannabinol, camphene, cinnamic acid, cinnamyl alcohol, dextrose, deoxyribose, EDTA, erucic acid, ethyl 2-trans-4-cis-decadienoate, eucalyptol, fructose, gallic acid, glutamic acid, glutamine, glycinic acid, indic acid, isoamyl alcohol, oenanthic acid, pelargonic acid, phosphoric acid, phytic acid, proline, lactose, linalool, maltose, mandelic acid, mangoferin, manzanate, menthol, myrcene, nerol, nervonic acid, nicotinic acid, nucleosidic acid, mannitol, pectin, limonene, pinene, punicic acid, ribose, sabinene, sativic acid, sebacic acid, sorbitol, sucralose, tocopherols, thiamine, thymol, vanillin, xylose, xylitol and ethyl maltol.

In the foregoing description, the invention is described with reference to specific exemplary embodiments thereof. It will, however, be evident that various modifications and changes may be made thereto without departing from the broader spirit and scope of the invention as set forth in the appended claims.

What is claimed is:

1. A method for delivering a drug to a user comprising:
an electronic cigarette wherein the electronic cigarette comprises a liquid formulation comprising:
at least one drug; and
at least one biologically acceptable carrier;
wherein the at least one drug is formed with an acid or alcohol and has a first vapor pressure at a first temperature; and
wherein the liquid formulation is heated by a heating element resulting in the generation of an aerosol suitable for inhaling by the user.

2. The method of claim 1, wherein the at least one drug is nicotine.

3. The method of claim 1, wherein the at least one drug is cannabinol.

4. The method of claim 1, wherein the at least one drug is associated with a nicotine replacement therapy.

5. The method of claim 1, wherein the at least one drug is associated with a medication-assisted therapy.

6. The method of claim 1, wherein the at least one drug is salt-based.

7. The method of claim 1, wherein the first vapor pressure is less than 20 millimeters of mercury (mmHg).

8. The method of claim 1, wherein the first temperature is approximately 200 degrees Celsius.

9. The method of claim 1, wherein the at least first drug further comprises a first melting point and a first boiling point and wherein the difference between the first melting point and the first boiling point is at least 50 degrees Celsius.

10. The method of claim 9, wherein the heating element operates at a first operating temperature and the first boiling point of the first drug is less than 40 degrees Celsius lower than the operating temperature.

11. A liquid storage device comprising:
a housing suitable for coupling with an electronic cigarette wherein the coupling provider thermal communication with a heating element within the electronic cigarette;
a liquid formulation comprising:
at least one drug; and
at least one biologically acceptable carrier;
wherein the at least one drug is formed with an acid or alcohol and has a first vapor pressure at a first temperature; and
wherein the liquid formulation is heated by the heating element resulting in the generation of an aerosol suitable for inhaling by a user.

12. The device of claim 11, wherein the at least one drug is nicotine.

13. The device of claim 11, wherein the at least one drug is cannabinol.

14. The device of claim 11, wherein the at least one drug is associated with a nicotine replacement therapy.

15. The device of claim 11, wherein the at least one drug is associated with a medication-assisted therapy.

16. The device of claim 11, wherein the at least one drug is salt-based.

17. The device of claim 11, wherein the first vapor pressure is less than 20 millimeters of mercury (mmHg).

18. The device of claim 11, wherein the first temperature is approximately 200 degrees Celsius.

19. The device of claim 11, wherein the at least first drug further comprises a first melting point and a first boiling point and wherein the difference between the first melting point and the first boiling point is at least 50 degrees Celsius.

20. The device of claim 19, wherein the heating element operates at a first operating temperature and the first boiling point of the first drug is less than 40 degrees Celsius lower than the operating temperature.
